(19) United States
(12) Patent Application Publication
(10) Pub. No.: US 2020/0219307 A1
(43) Pub. Date: Jul. 9, 2020

(54) SYSTEM AND METHOD FOR CO-REGISTRATION OF SENSORS

(71) Applicants: Adam Bartsch, Chaska, MN (US); Rajiv Dama, Chanhassen, MN (US)

(72) Inventors: Adam Bartsch, Chaska, MN (US); Rajiv Dama, Chanhassen, MN (US)

(21) Appl. No.: 16/737,325

(22) Filed: Jan. 8, 2020

Related U.S. Application Data
(60) Provisional application No. 62/789,849, filed on Jan. 8, 2019.

(51) Int. Cl. G06T 15/20; G01P 21/00; G06T 7/73; G06T 7/00; A61B 5/00

(52) U.S. Cl. CPC G06T 15/205; G01P 21/00; A61B 5/6814; G06T 7/0012; G06T 7/73

(57) ABSTRACT

A method of co-registration of a plurality sensors configured for sensing impact to a body part of a user may include establishing a location and an orientation of the plurality of sensors relative to one another and establishing a location and an orientation of the plurality of sensors relative to an anatomical feature of the body part. Establishing a location and an orientation of the plurality of sensors relative to one another may be performed using a 2D image or analytically by analyzing sensor results.

### CROSS-REFERENCE TO RELATED APPLICATIONS

The present application claims priority to U.S. Provisional Application No. 62/789,849 entitled Impact Sensing and Co-Registration, and filed on Jan. 8, 2019, the content of each of which is hereby incorporated by reference herein in its entirety.

### TECHNOLOGICAL FIELD

The present disclosure relates to devices and systems for impact sensing and assessment. More particularly, the present disclosure relates to co-registering the sensors used for impact sensing. Still more particularly, the present application relates to particular methods of co-registration of sensors.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Researchers and product developers have long since been trying to accurately and precisely sense impacts such as head impacts or other motion data occurring during sports, military activities, exercise, or other activities. While the ability to sense impacts has been available for some time, the ability to sense impacts with sufficient accuracy and precision to provide meaningful results has been more elusive. In the case of head impacts, the road blocks preventing such accuracy and precision include relative movement between the sensors and the head, false positive data, insufficient processing power and processing speed on a wearable device, and a host of other difficulties.

One solution to the relative movement issues has been to rely on a mouthguard that couples tightly with the upper teeth of a user and, as such, is relatively rigidly tied to the skull of a user. However, without knowledge of the sensor positions relative to the relevant aspects of a user's anatomy or knowledge of the sensor positions relative to one another, the precision and accuracy may be limited.

### SUMMARY

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

In one or more embodiments, a method of co-registration of a plurality sensors configured for sensing impact to a body part of a user may be provided. The method may include establishing a location and an orientation of the plurality of sensors relative to one another and establishing a location and an orientation of the plurality of sensors relative to an anatomical feature of the body part. In one or more embodiments, establishing a location and an orientation of the plurality of sensors relative to one another may be performed using a 2D image.

In one or more embodiments, a method of co-registration of a plurality of impact sensors configured for sensing the impact to a body part of a user may include establishing the location and the orientation of the plurality of sensors relative to one another. In one or more embodiments, establishing the location and the orientation of the plurality of sensors relative to one another may be performed analytically by analyzing sensor results.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1 is a front view of model experiencing an impact on a model of a head, according to one or more embodiments.

FIG. 2 is a still frame of footage of a player experiencing a head impact.

FIG. 3 is perspective view of a mouthpiece in place on a user and showing relative positions and orientations of the impact sensors relative to an anatomical feature or landmark of the user, according to one or more embodiments.

FIG. 4 is a diagram of a method of co-registering impact sensors, according to one or more embodiments.

FIG. 5 is a diagram of a method of co-registering impact sensors, according to one or more embodiments.

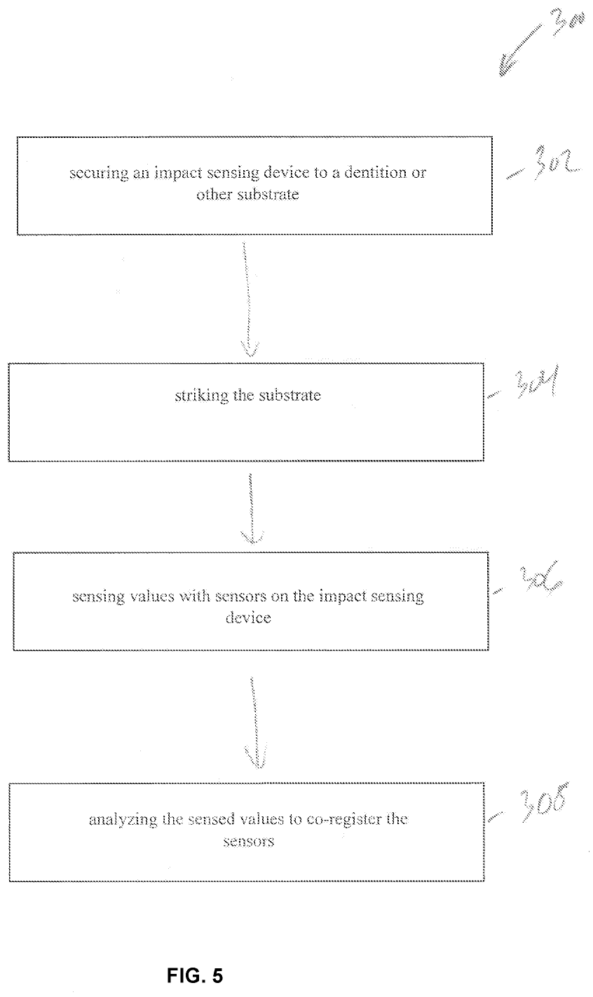

### DETAILED DESCRIPTION

The present disclosure, in one or more embodiments, relates to using sensors to sense impacts to a user and more particularly to methods of co-registering the sensors to provide highly accurate and precise results. Co-registration may involve determining the sensor locations and orientations relative to one another as well as relative to particular anatomical features of a user. In one or more embodiments, 2D imaging may be used allowing for relatively conventional devices to co-register sensors. Alternatively or additionally, analytical processes may be used that, again, allow for relatively conventional devices to establish co-registration. The co-registered devices may allow for more accurately and precisely establishing impact locations and direction and the effects of impacts at other locations on the user's body. In one or more embodiments, determining the kinematic and/or resulting forces at the center of gravity of a user's head may be determined and used to assess the effect of the impact on the user.

Before getting into the details of co-registration, the present application is based on the presence of an impact sensing device having one or more sensors. One example of an impact sensing device is a mouthguard equipped with sensors that may be properly coupled to a user's upper jaw via the upper teeth. In one or more embodiments, a mouthguard may be provided that is manufactured according to the methods and systems described in U.S. patent application Ser. No. 16/682,656, entitled Impact Sensing Mouthguard, and filed on Nov. 13, 2019, the content of which is hereby incorporated by reference herein in its entirety.

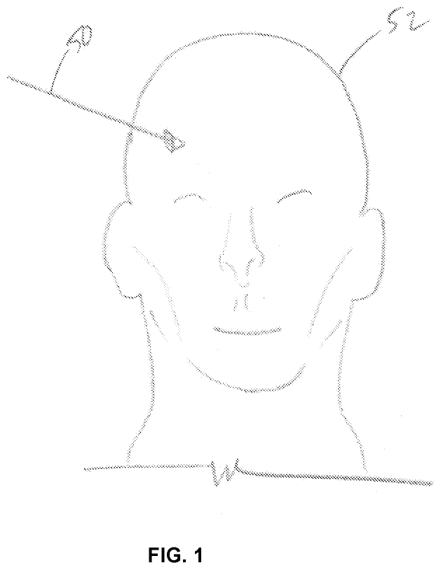

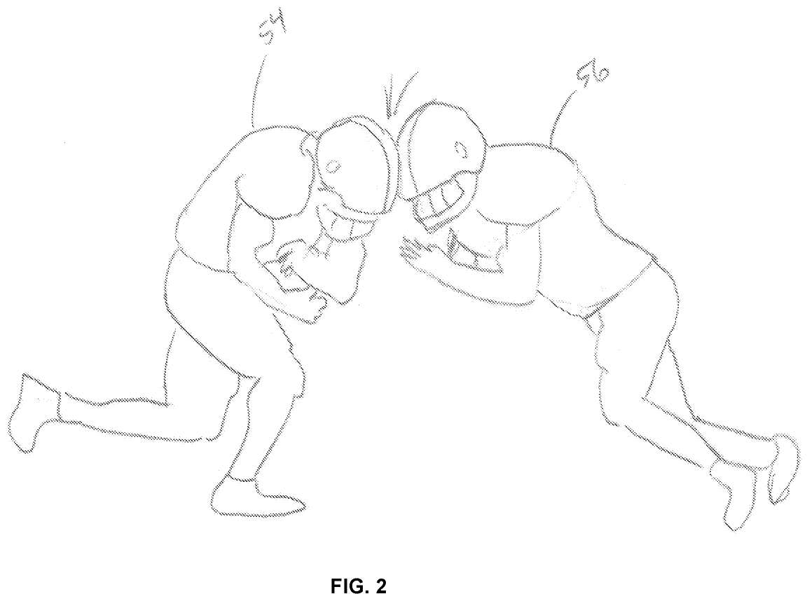

Turning now to FIGS. 1-3, a series of figures are provided for an understanding of co-registration. As shown in FIG. 1, a user may experience a blow (depicted by vector 50) to the head 52. For example, in an American Football game, head collisions as shown in FIG. 2 may be quite common. FIG. 2 shows a still frame example of video footage of an impact. As shown, a ball carrier 54 in a football game has lowered his head to brace for impact of an oncoming defensive player 56. As shown, the helmets of the two players create an impact to both players. The impact is to the left/front side of the ball carrier's helmet and to the right/front side of the defensive player's helmet. The resultant force vector shown in FIG. 1 may be determined from sensors on the defensive player 56. Moreover, the effects of the impact may be determined at or near the center of gravity of the head such that the effects of impact on the brain may be assessed. The resulting force and/or kinematics determined from the sensors on an impact sensing device may be more accurate and precise if the relative positions/orientations of the sensors are known and if the positions/orientations of the sensors relative to the head are known.

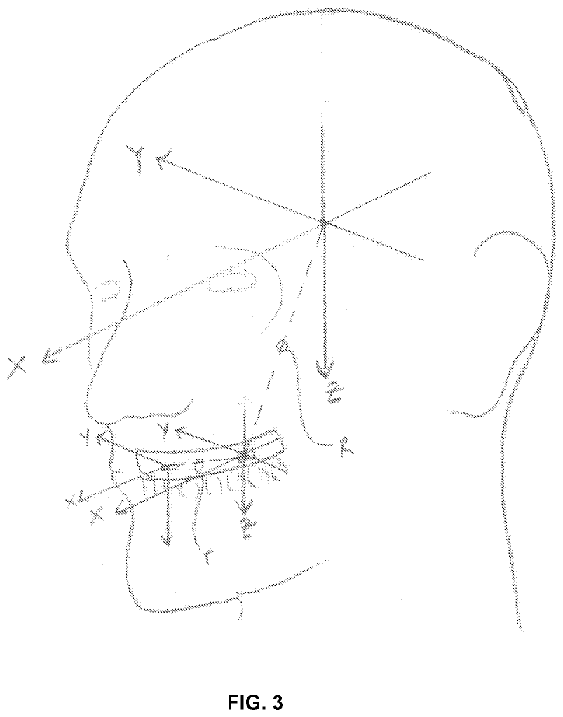

FIG. 3 shows a diagram of two sensors arranged on a mouthguard within a user's mouth. FIG. 3 also shows the respective local axes of the sensors and the user axes based on anatomical features. While the sensors may be arranged on three orthogonal axes and while the respective axes shown in FIG. 3 appear to be generally parallel, this may not always be the case. Moreover, while the sensors may be adapted to sense accelerations along and/or about their respective axes, the sensors may not always be perfectly placed and obtaining data defining the relative position and orientation of the sensors relative to one another may be helpful. Moreover, while the sensors' positions relative to the center of gravity of a head or other anatomical landmark of the user may be generally known or assumed, a more precise dimensional relationship may allow for more precise analysis. Depending on the demands on the accuracy of the impact data, co-registration may be very advantageous. For example, calculated impact kinematics may vary 5-15% where co-registration is not performed. In one or more embodiments, where user anthropometry is relatively consistent across a group of users and assumptions about the anthropometry is used, the errors may be reduced to 5-10% where co-registration is performed based on the assumptions. For example, where a true impact results in a 50 g acceleration, the measured impact may be 45 g to 55 g. Where user-specific anthropometry is used, the errors may be further reduced.

In one or more embodiments, co-registration may be performed by measuring. For example, measuring may include physically measuring the sensor position relative to user anatomy such as described in U.S. Pat. No. 9,585,619 entitled registration of head impact detection assembly, and filed on Feb. 17, 2012, the content of which is hereby incorporated by reference here in its entirety. In one or more embodiments, measuring may include directly measuring the positions and orientations using an internal scanning device or indirectly measuring the positions and orientations using multiple scans (e.g., one of the user and one of the device where the data is tied together with markers). For example, in one or more embodiments, co-registration may be performed using magnetic resonance imaging (MRI) or computerized tomography (CT) as described in U.S. patent application Ser. No. 16/720,589 entitled Methods for Sensing and Analyzing Impacts and Performing an Assessment, and filed on Dec. 19, 2019, the content of which is hereby incorporated by reference herein in its entirety. Still other internal scanning devices may be used.

In one or more embodiments, two-dimensional (2D) imaging may be used to establish co-registration values. That is, for example, a point cloud of data may be captured using a single 2D image in the coronal plane (e.g., front view) or sagittal plane (e.g., profile view), or from a series of 2D images stitched together into a 3D rendering such as is done with facial recognition technology. This may be performed with the mouthguard alone and/or with the mouthguard in the mouth of a user, for example, with the mouth open wide or at least slightly open such that particular positions and/or orientations of the sensors on the mouthguard may be identified as well as the head geometry. The information captured may allow the sensor positions and orientations relative to one another to be identified as well as the sensor positions and orientations relative to the user's head. It is to be appreciated that 2D imagery may involve issues of scale depending on whether the photo is a close-up view or a more distant view. Known features on the sensing device with known separation distances may be used to calibrate the image, so to speak, and allow it to be used for measurements that are to scale. In other embodiments a reference tool may be included in the image such as a ruler or other device with a known size. In still other embodiments, bodily features may be measured and used as an input to assist with adjusting the scale of the image.

A photograph or other image (e.g., without or aside from facial recognition technology) may also be used for purposes of co-registration. In one or more embodiments, a photograph of the user and/or a photograph of the mouth guard in place in the mouth of the user and with other features of the users face present in the photo may be used to establish co-registration. The image may be a front view, profile view, or a perspective view, may be used. In one or more embodiments, a selfie may be taken of the user with the user's mouth open and/or closed and the data from the image may be used to establish co-registration. Still other approaches to capturing the relative position and orientation of the sensor relative to one another and the relative position and orientation of the sensor relative to the head of the user may be provided. Calibration or scaling techniques mentioned above may be used in this context as well.

Using one or more of the above-referenced technologies or approaches co-registration of the sensors used to sense impacts may be performed. Co-registration may be performed for purposes of establishing spatial relationships between one or more sensors and, as such, may include establishing distances between sensors and relative positions as well as establishing relative orientations of sensors. Co-registration may also be performed for purposes of establishing spatial relationships of the one or more sensors relative to the head or particular structures of the head or other human body part. As such, co-registration may include establishing distances between one or more sensors and a particular body part or portion of the body and may also include establishing relative orientations of the one or more sensors relative to the body. The particular locations, orientations, and relative locations and orientations can be useful to reduce and/or eliminate error due to unknown, inaccurate, or imprecise sensor locations and orientations.

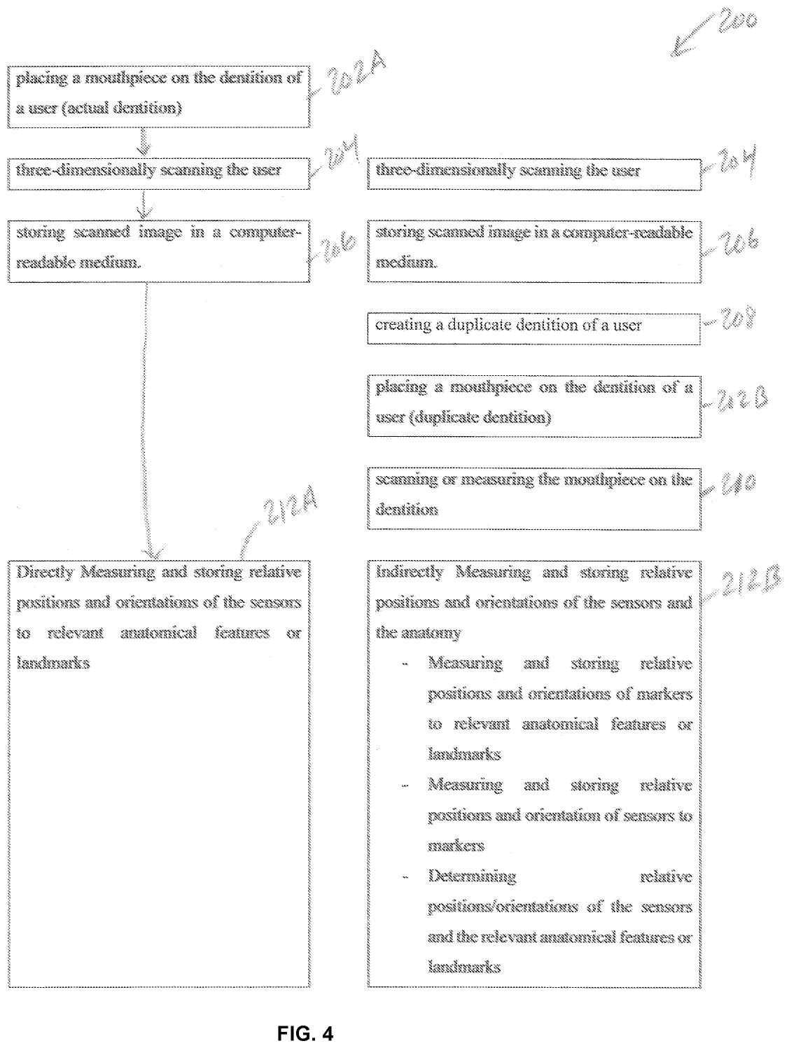

In one or more embodiments, and with reference to FIG. 4, a method 200 of co-registration may be provided. The method 200 may include placing a mouthpiece on a dentition of a user (202A/202B). In one or more embodiments, this step may include placing the mouthpiece in the user's mouth (202A). Alternatively or additionally, placing the mouthpiece on a dentition of the user may include placing the mouthpiece on a duplicate dentition of the mouth of a user (202B). The method may also include obtaining one or more two-dimensional images of the user (204). This step may be performed with the mouthpiece in place in the user's mouth or without the mouthpiece in the mouth of the user. In either case, the obtained image may be stored in a computer-readable medium (206).

Where the mouthpiece is in the mouth during imaging, the relative positions and orientations of sensors and anatomy may be measured and stored directly (212A). For example, and as shown in FIG. 3, the relative positions (r) and orientations of the sensors may be ascertained from the image to verify, adjust, or refine the relative positions and orientations of the sensors relative to one another. It is to be appreciated that where the actual mouthguard is being used during image capture, manufacturing tolerances associated with sensor placement may be accounted for during co-registration by measuring the actual position and orientation of the sensors. Moreover, and with respect to direct measurement of sensor positions, the images may be used to measure the positions and orientations of the sensors relative to particular anatomical features or landmarks. For example, in one or more embodiments, the relative position (R) of the sensors and the relative orientation of the sensors with respect to the center of gravity of the head or with respect to particular portions of the brain may be measured and stored. That is, for example, with a profile image and knowledge of the center of gravity of a user's head with respect to the ear and eye, the relative position of the sensor may be established with respect to the center of gravity of the head.

Where the mouthpiece is not in the mouth during scanning, the relative positions and orientations of sensors and anatomy may be measured and stored indirectly (212B). That is, the relative positions of markers on the anatomy may be stored based on the scan of the user. For example, marker locations on the user's teeth relative to particular anatomical features or landmarks such as the center of gravity of the head may be stored. Further, where the mouthpiece is not placed in the mouth during the scan of the user, the method may include creating a duplicate dentition of the user's mouth. (208) This may be created from a MRI/CT scan using a 3-dimensional printer, using bite wax impressions, or using other known mouth molding techniques. The mouthpiece may be placed on the duplicate dentition and physical measurements of the sensors relative to markers on the dentition may be taken. (210) Additionally or alternatively, scans such as laser scans, two-dimensional images or point cloud images, MRI scans, CT scans or other scans of the mouthpiece on the duplicate dentition may be used to identify the sensor locations relative to the markers on the dentition. (210) The markers on the duplicate dentition may coincide with the markers used in the imaging of the user. As such, the method may include indirectly determining the positions and orientations of the sensors relative to the anatomical features or landmarks of interest, such as the center of gravity of the head, by relying on the markers tying the two sets of data together. (212B)

In the above method, while particular levels of co-registration may be established using a single 2D image, multiple 2D images may be helpful to further refine the results. As such, in one or more embodiments, multiple 2D images may be used to further establish relative positions and orientations of sensors relative to one another and relative to particular portions of a user's anatomy.

In the above method, system knowledge of human anatomy together with pixel lightness or darkness or colors variations may allow the system to identify particular anatomical features or sensors within the image. This may be particularly true where the images that are captured are categorized in a particular way such that the system knows, for example, that it is analyzing a profile view, front view, or a perspective view. As such, anatomical feature identification and sensor identification may occur automatically.

Additionally or alternatively, user input may be used to further refine the results. For example, a user may access a photo after image capture or the user may be automatically prompted with the image to provide further input. The input may allow the user to identify particular portions of the image as particular bodily features or particular portions of a sensing device as sensor locations, for example. Orientations of the sensors or the body parts may also be subject to further input. For example, and as to locations, a user may be prompted with a cross-hair to place on the image at particular locations such as the eye, the ear, a sensor location, or another relevant location. The cross-hair may assist the system in knowing more particularly where a feature or item is within in an image. Regarding orientations, a set of vertices may be provided allowing a user to adjust the angle of a set of axes relative to a sensor location. That is, where a sensor is canted from vertical in an image, for example, the user may be provided with vertices to align the vertices with the angle or direction of the sensor. Still other user input may be provided to augment the information obtained from the image.

In still other embodiments, data analysis may assist in determining relative location and/or orientation. For example, differing sensed forces and/or accelerations may be analyzed to establish relative location and orientation of the sensor with respect to one another. In one or more embodiments, an impact may be imparted on the mouth guard or other sensing device along or about a known axis and/or with other known parameters. The sensed impact and/or acceleration of the several sensors of the device may then be analyzed to back calculate their location and orientation.

In one or more embodiments, uni-axial motion may be imparted on the sensing device. That is, a linear acceleration along a single axis may be imparted on the sensing device. Alternatively or additionally, a rotation about a single axis may be imparted on the sensing device. One or more separate uni-axial motions may be imparted on the device to provide more data with respect to the position and orientation of the sensors.

Uni-axial motion may include accelerations, velocities, or displacements, for example. In one embodiment, an impact sensing device may be dropped along a vertical axis, for example, and the data sensed by each sensor (either due to the dropping acceleration or the impact acceleration at the bottom of travel or both) may be analyzed to determine orientation. That is, to the extent the sensors are not arranged orthogonally to the vertical axis, sensor results that are non-zero may be received along axes that were thought to be orthogonal to the vertical axis. These results may be used to define how canted the sensor is in one or more directions (e.g., about the x or y axes shown in FIG. 3). Additionally or alternatively, the impact sensing device may be rotated about an axis and the several sensor results may be used to determine the distance from the rotation axis, for example. In one or more embodiments, uni-axial motion (e.g., linear or rotation) may be used along a series of axes to assist with determining locations and orientations of the sensors. In one embodiment, uni-axial motion may be imparted separately along each of three orthogonal axes and about each of three orthogonal axes.

In one or more embodiments, a method 300 of co-registration may include securing an impact sensing device to a dentition or other substrate. (302) In one or more embodiments, the dentition may include a duplicate dentition, a form, or other substrate that the impact sensing may be adequately coupled to. In some embodiments, the substrate may be a device for securing the impact sensing device to or resting the impact sensing device on a motion-controlled holder or platform. The method may also include striking dropping, turning, spinning, or otherwise accelerating, displacing, or otherwise moving the substrate (304). Moving the substrate may be performed uni-axially such as a single linear motion or a single rotational motion. In one or more embodiments, the motion may be along or about an assumed axis of one or more of the sensors. The method may also include sensing values with sensors on the impact sensing device (306). The method may also include analyzing the sensed values to co-register the sensors (308). As mentioned, in the case of uni-axial linear motion, several axes may be isolated or attempted to be isolated such that value sensed along those axes may help determine an orientation of a sensor. Also, in the case of uni-axial rotational motion, the effect of rotation on the sensor may assist with determining how close or far from the rotational axis, a particular sensor is. By using isolated uni-axial rotation about multiple axes, the 3D position of the sensors may be determined.

Aside from and beyond co-registration, the co-registered impact sensing device may be used to sense impacts to a user and develop impact data. The impact data may be analyzed to determine kinematics, forces, or other values at or near the sensed location, at particular points of interest in the head (e.g., head center of gravity), or at other locations. In one or more embodiments, rigid body equations or deformable body equations may be used such as those outlined in U.S. Pat. Nos. 9,289,176, 9,044,198, 9,149,227, and 9,585,619, the content of each of which is hereby incorporated by reference herein in its entirety.

It is to be appreciated that many methods have been described herein that may have logical and practical application on a computer chip or other circuitry embedded in a mouthguard, other oral appliance, or another device capable of adequate coupling with the head of a user or other portion of a user. As such, while the methods have been described as such, many of the methods may be suitable as part of a sophisticated mouthguard or smart mouthguard, for example. That is, with very few exceptions, any of the methods described herein may be part of the circuitry of a mouthguard or other oral appliance. The exceptions may be methods involving other equipment such as MRI equipment, CT equipment, scanners, or other equipment not embeddable in an oral appliance. Other exceptions may include methods that simply are not programmable and require human performance or, as mentioned, performance of other equipment. Nonetheless, even when other equipment is used to perform a method, particular parts of pieces of the method may be part of the mouthguard.

For purposes of this disclosure, any system described herein may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, a system or any portion thereof may be a minicomputer, mainframe computer, personal computer (e.g., desktop or laptop), tablet computer, embedded computer, mobile device (e.g., personal digital assistant (PDA) or smart phone) or other hand-held computing device, server (e.g., blade server or rack server), a network storage device, or any other suitable device or combination of devices and may vary in size, shape, performance, functionality, and price. A system may include volatile memory (e.g., random access memory (RAM)), one or more processing resources such as a central processing unit (CPU) or hardware or software control logic, ROM, and/or other types of nonvolatile memory (e.g., EPROM, EEPROM, etc.). A basic input/output system (BIOS) can be stored in the non-volatile memory (e.g., ROM), and may include basic routines facilitating communication of data and signals between components within the system. The volatile memory may additionally include a high-speed RAM, such as static RAM for caching data.

Additional components of a system may include one or more disk drives or one or more mass storage devices, one or more network ports for communicating with external devices as well as various input and output (I/O) devices, such as digital and analog general purpose I/O, a keyboard, a mouse, touchscreen and/or a video display. Mass storage devices may include, but are not limited to, a hard disk drive, floppy disk drive, CD-ROM drive, smart drive, flash drive, or other types of non-volatile data storage, a plurality of storage devices, a storage subsystem, or any combination of storage devices. A storage interface may be provided for interfacing with mass storage devices, for example, a storage subsystem. The storage interface may include any suitable interface technology, such as EIDE, ATA, SATA, and IEEE 1394. A system may include what is referred to as a user interface for interacting with the system, which may generally include a display, mouse or other cursor control device, keyboard, button, touchpad, touch screen, stylus, remote control (such as an infrared remote control), microphone, camera, video recorder, gesture systems (e.g., eye movement, head movement, etc.), speaker, LED, light, joystick, game pad, switch, buzzer, bell, and/or other user input/output device for communicating with one or more users or for entering information into the system. These and other devices for interacting with the system may be connected to the system through I/O device interface(s) via a system bus, but can be connected by other interfaces such as a parallel port, IEEE 1394 serial port, a game port, a USB port, an IR interface, etc. Output devices may include any type of device for presenting information to a user, including but not limited to, a computer monitor, flat-screen display, or other visual display, a printer, and/or speakers or any other device for providing information in audio form, such as a telephone, a plurality of output devices, or any combination of output devices.

A system may also include one or more buses operable to transmit communications between the various hardware components. A system bus may be any of several types of bus structure that can further interconnect, for example, to a memory bus (with or without a memory controller) and/or a peripheral bus (e.g., PCI, PCIe, AGP, LPC, I2C, SPI, USB, etc.) using any of a variety of commercially available bus architectures.

One or more programs or applications, such as a web browser and/or other executable applications, may be stored in one or more of the system data storage devices. Generally, programs may include routines, methods, data structures, other software components, etc., that perform particular tasks or implement particular abstract data types. Programs or applications may be loaded in part or in whole into a main memory or processor during execution by the processor. One or more processors may execute applications or programs to run systems or methods of the present disclosure, or portions thereof, stored as executable programs or program code in the memory, or received from the Internet or other network. Any commercial or freeware web browser or other application capable of retrieving content from a network and displaying pages or screens may be used. In some embodiments, a customized application may be used to access, display, and update information. A user may interact with the system, programs, and data stored thereon or accessible thereto using any one or more of the input and output devices described above.

A system of the present disclosure can operate in a networked environment using logical connections via a wired and/or wireless communications subsystem to one or more networks and/or other computers. Other computers can include, but are not limited to, workstations, servers, routers, personal computers, microprocessor-based entertainment appliances, peer devices, or other common network nodes, and may generally include many or all of the elements described above. Logical connections may include wired and/or wireless connectivity to a local area network (LAN), a wide area network (WAN), hotspot, a global communications network, such as the Internet, and so on. The system may be operable to communicate with wired and/or wireless devices or other processing entities using, for example, radio technologies, such as the IEEE 802.xx family of standards, and includes at least Wi-Fi (wireless fidelity), WiMax, and Bluetooth wireless technologies. Communications can be made via a predefined structure as with a conventional network or via an ad hoc communication between at least two devices.

Hardware and software components of the present disclosure, as discussed herein, may be integral portions of a single computer, server, controller, or message sign, or may be connected parts of a computer network. The hardware and software components may be located within a single location or, in other embodiments, portions of the hardware and software components may be divided among a plurality of locations and connected directly or through a global computer information network, such as the Internet. Accordingly, aspects of the various embodiments of the present disclosure can be practiced in distributed computing environments where certain tasks are performed by remote processing devices that are linked through a communications network. In such a distributed computing environment, program modules may be located in local and/or remote storage and/or memory systems.

As will be appreciated by one of skill in the art, the various embodiments of the present disclosure may be embodied as a method (including, for example, a computer-implemented process, a business process, and/or any other process), apparatus (including, for example, a system, machine, device, computer program product, and/or the like), or a combination of the foregoing. Accordingly, embodiments of the present disclosure may take the form of an entirely hardware embodiment, an entirely software embodiment (including firmware, middleware, microcode, hardware description languages, etc.), or an embodiment combining software and hardware aspects. Furthermore, embodiments of the present disclosure may take the form of a computer program product on a computer-readable medium or computer-readable storage medium, having computer-executable program code embodied in the medium, that define processes or methods described herein. A processor or processors may perform the necessary tasks defined by the computer-executable program code. Computer-executable program code for carrying out operations of embodiments of the present disclosure may be written in an object oriented, scripted or unscripted programming language such as Java, Perl, PHP, Visual Basic, Smalltalk, C++, or the like. However, the computer program code for carrying out operations of embodiments of the present disclosure may also be written in conventional procedural programming languages, such as the C programming language or similar programming languages. A code segment may represent a procedure, a function, a subprogram, a program, a routine, a subroutine, a module, an obj ect, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

In the context of this document, a computer readable medium may be any medium that can contain, store, communicate, or transport the program for use by or in connection with the systems disclosed herein. The computer-executable program code may be transmitted using any appropriate medium, including but not limited to the Internet, optical fiber cable, radio frequency (RF) signals or other wireless signals, or other mediums. The computer readable medium may be, for example but is not limited to, an electronic, magnetic, optical, electromagnetic, infrared, or semiconductor system, apparatus, or device. More specific examples of suitable computer readable medium include, but are not limited to, an electrical connection having one or more wires or a tangible storage medium such as a portable computer diskette, a hard disk, a random access memory (RAM), a read-only memory (ROM), an erasable programmable read-only memory (EPROM or Flash memory), a compact disc read-only memory (CD-ROM), or other optical or magnetic storage device. Computer-readable media includes, but is not to be confused with, computer-readable storage medium, which is intended to cover all physical, non-transitory, or similar embodiments of computer-readable media.

Various embodiments of the present disclosure may be described herein with reference to flowchart illustrations and/or block diagrams of methods, apparatus (systems), and computer program products. It is understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks. Alternatively, computer program implemented steps or acts may be combined with operator or human implemented steps or acts in order to carry out an embodiment of the invention.

Additionally, although a flowchart or block diagram may illustrate a method as comprising sequential steps or a process as having a particular order of operations, many of the steps or operations in the flowchart(s) or block diagram(s) illustrated herein can be performed in parallel or concurrently, and the flowchart(s) or block diagram(s) should be read in the context of the various embodiments of the present disclosure. In addition, the order of the method steps or process operations illustrated in a flowchart or block diagram may be rearranged for some embodiments. Similarly, a method or process illustrated in a flow chart or block diagram could have additional steps or operations not included therein or fewer steps or operations than those shown. Moreover, a method step may correspond to a method, a function, a procedure, a subroutine, a subprogram, etc.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1. A method of co-registration of a plurality sensors configured for sensing impact to a body part of a user, comprising:
establishing a location and an orientation of the plurality of sensors relative to one another; and
establishing a location and an orientation of the plurality of sensors relative to an anatomical feature of the body part,
wherein establishing a location and an orientation of the plurality of sensors relative to one another is performed using a 2D image.

2. The method of claim 1, wherein establishing a location and an orientation of the plurality of sensors relative to an anatomical feature of the body part is performed using a 2D image.

3. The method of co-registration of claim 2, wherein the 2D image comprises a front view and a profile view.

4. The method of co-registration of claim 2, wherein the 2D image comprises a series of 2D images stitched together into a 3D rendering.

5. The method of co-registration of claim 2, wherein the method comprises automatically identifying the anatomical feature in the 2D image.

6. The method of co-registration of claim 2, wherein the method comprises receiving user input identifying a location of the anatomical feature in the 2D image.

7. The method of co-registration of claim 2, wherein the method comprises receiving user input identifying an orientation of the anatomical feature in the 2D image.

8. The method of claim 1, wherein establishing a location and an orientation of the plurality of sensors relative to an anatomical feature is performed directly.

9. The method of claim 1, wherein establishing a location and an orientation of the plurality of sensors relative to an anatomical feature is performed indirectly.

10. The method of claim 9, wherein the method relies on markers on a dentition that coincide with markers used in an image of the user.

11. A method of co-registration of a plurality of impact sensors configured for sensing the impact to a body part of a user, comprising:
establishing the location and the orientation of the plurality of sensors relative to one another, wherein establishing the location and the orientation of the plurality of sensors relative to one another is performed analytically by analyzing sensor results.

12. The method of co-registration of claim 11, wherein the sensor results are based on a uni-axial test.

13. The method of co-registration of claim 12, wherein the uni-axial test is a linear drop test along a single axis.

14. The method of co-registration of claim 12, wherein the uni-axial test is a rotational test about a single axis.

15. The method of co-registration of claim 11, wherein the uni-axial test comprises a linear test and a rotational test along and about, respectively, each of three orthogonal axes.
